(19) United States
(12) Patent Application Publication
(10) Pub. No.: US 2021/0298992 A1
(43) Pub. Date: Sep. 30, 2021

(54) ATOMIZING DEVICE FOR A STEAMING FACE INSTRUMENT

(71) Applicant: Shenzhen Aifan Technology Co., Ltd., Shenzhen (CN)

(72) Inventor: Jianping Liu, Heyuan (CN)

(21) Appl. No.: 16/931,363

(22) Filed: Jul. 16, 2020

(30) Foreign Application Priority Data
Mar. 27, 2020 (CN) 202020430489.7

(51) Int. Cl. A61H 33/12; A61H 33/06; A61M 11/00

(52) U.S. Cl. CPC A61H 33/12; A61H 33/063; A61H 2201/0228; A61H 2033/068; A61M 11/005

(57) ABSTRACT

An atomizing device for a steaming face instrument, which comprises: a water delivery channel, the water delivery channel includes a water inlet and a water outlet; an atomizing sheet, the atomizing sheet is arranged at the water outlet , for atomizing water; and a heating mechanism, the heating mechanism is connected to the water outlet for heating the water mist, so that the temperature of the water mist is controlled between 45 and 60 degrees. By adopting the above design, the disclosure enables the liquid water to be atomized by the high-frequency resonance of the atomizing sheet, and then heats the water mist, so that the temperature of the water mist is controlled at 45-60 degrees, which is safe and effective. The volume of the face steaming instrument is reduced, it is convenient to carry, its power consumption is small, energy saving and environmental protection.

### CROSS-REFERENCES TO RELATED APPLICATIONS

This application claims the priority of Chinese patent application no. 202020430489.7, filed on Mar. 27, 2020, the entire contents of all of which are incorporated herein by reference.

### FIELD

The present utility model relates to the technical field of a steaming face instrument, and more particularly, to an atomizing device for a steaming face instrument.

### BACKGROUND

At present, a steaming face instrument on the market uses PTC heating, which boils water beyond the boiling point of water, and then allows water to enter the gas state from the liquid state, changing the physical structure of water. Due to high temperature, poor control of the distance can easily cause burns. Moreover, this steaming face instrument is bulky and inconvenient to carry, so it must be placed on a table or the like before it can be used.

Therefore, there is an urgent need to design an atomizing device that can solve one or more of the above problems.

### BRIEF SUMMARY OF THE DISCLOSURE

The technical problem to be solved in the disclosure, aiming at the defects of the prior art, provides an atomizing device for a steaming face instrument.

In order to achieve the above said targets, the technical solution of the disclosure is as follows: an atomizing device for a steaming face instrument, the atomizing device includes a water delivery channel, which includes a water inlet and a water outlet; an atomizing sheet provided at the water outlet for atomizing water; and a heating mechanism, which is connected to the water outlet, is used to heat the water mist, so that the temperature of the water mist is controlled between 45 and 60 degrees.

### BRIEF DESCRIPTION OF THE DRAWINGS

The following drawings are merely examples for illustrative purposes according to various disclosed embodiments and are not intended to limit the scope of the present disclosure.

FIG. 1 illustrates a schematic structural view consistent with the disclosed embodiments;

FIG. 2 illustrates a cross-sectional view consistent with the disclosed embodiments;

FIG. 3 illustrates an exploded view consistent with the disclosed embodiments;

FIG. 4 illustrates a schematic structural view of a water delivery channel consistent with the disclosed embodiments;

FIG. 5 illustrates a schematic structural view of a heat-insulating channel consistent with the disclosed embodiments.

In the picture:

Water delivery channel 1; Water inlet 11; Water outlet 12; Groove 121;

Insulation channel 2; Convex ring 21;

Atomizing sheet 3;

Heating tube 4;

Heat source 5.

### DETAILED DESCRIPTION

In order to make the above objects, features and advantages of the disclosure more obvious and easy to understand, the following describes the specific implementation of the present disclosure in detail with reference to the drawings. In the following description, many specific details are set forth in order to fully understand the present disclosure. However, the present disclosure can be implemented in many other ways than those described here. Those skilled in the art can make similar improvements without violating the connotation of the present disclosure. Therefore, the present disclosure is not subject to limit.

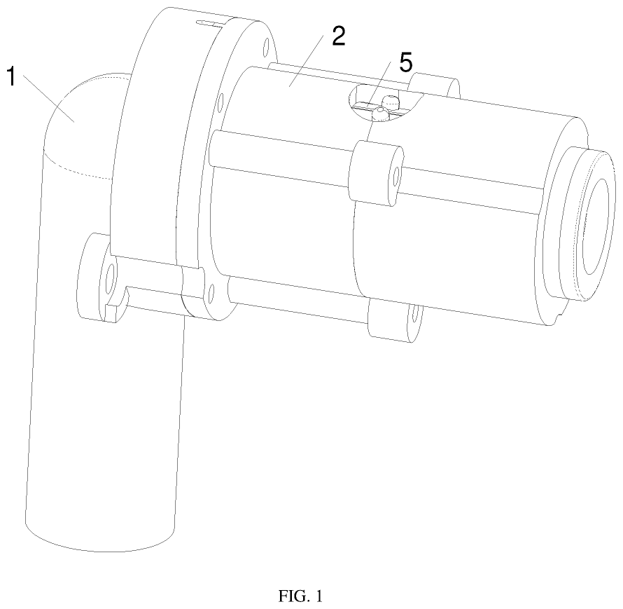

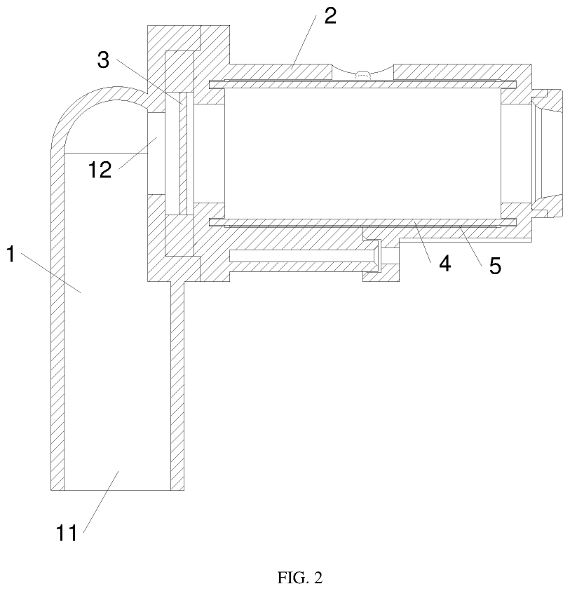

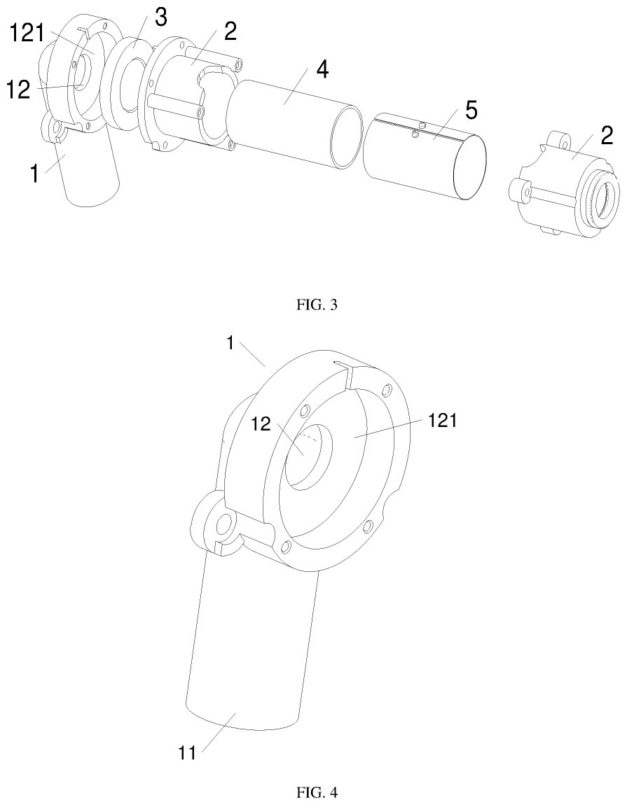

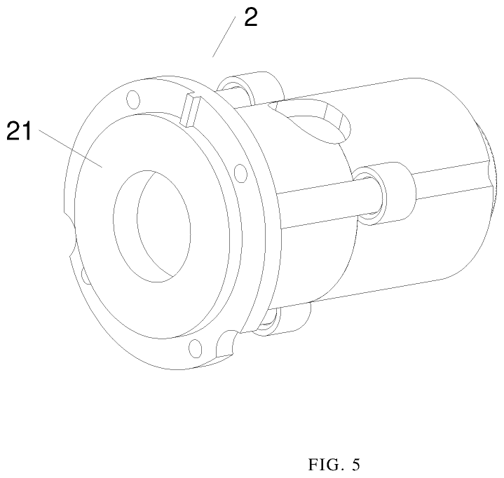

As shown in FIGS. 1-5, the present disclosure provides an atomizing device for a steaming face instrument. The atomizing device includes:

Water delivery channel 1, the water delivery channel 1 includes a water inlet 11 and a water outlet 12;

An atomizing sheet 3, which is provided at the water outlet 12 for atomizing water; and

A heating mechanism, which is connected to the water outlet 12 and is used to heat the water mist to control the temperature of the water mist between 45 and 60 degrees.

In some embodiments, the water outlet 12 is provided with a groove 121, the atomizing sheet 3 is disposed at the groove 121, and the atomizing film 3 is coaxially arranged with the water outlet 12.

In some embodiments, the atomizing sheet 3 is an ultrasonic atomizing sheet 3.

In some embodiments, the heating mechanism includes a heat insulation channel 2, a heating tube 4, and a heat source 5, one end of the heat insulation channel 2 is connected to the water outlet 12, and the atomizing sheet 3 is fixed on the water outlet 12, another end of the heat insulation channel 2 is connected to the spout, the heating tube 4 is disposed in the heat insulation channel 2 and is coaxial with the water outlet 12, the heat source 5 surrounds the outer wall of the heating tube 4.

In some embodiments, a convex ring 21 is provided at an end of the heat-insulating channel 2 connected to the water outlet 12, the convex ring 21 cooperates with the groove 121 to fix the atomizing sheet 3, The convex ring 21 is arranged coaxially with the water outlet 12.

In some embodiments, the heating tube 4 is made of metal, and the length of the heating tube 4 is 20-30 mm.

In some embodiments, the heat source 5 is a heating film or a heating wire.

The beneficial effects of the disclosure are: compared with the prior art, the disclosure includes the water delivery channel, the water delivery channel includes the water inlet and the water outlet; the atomizing sheet, the atomizing sheet is disposed at the water outlet, the atomizing sheet is used for atomizing water; and a heating mechanism, which is connected to the water outlet and is used for heating the water mist, so that the temperature of the water mist is controlled between 45-60 degrees. By adopting the above design, the disclosure enables the liquid water to be atomized by the high-frequency resonance of the atomizing sheet, and then heats the water mist, so that the temperature of the water mist is controlled at 45-60 degrees, which is safe and effective. The volume of the steaming face instrument is reduced, it is convenient to carry, its power consumption is small, energy saving and environmental protection.

Specifically, water enters from the water inlet 11 and passes through the atomizing sheet 3 of the water outlet 12 to break up the liquid water molecular structure to generate a naturally flowing water mist. In this embodiment, the ultrasonic atomizing sheet 3 is used. The so-called ultrasonic atomizing sheet 3 uses electronic high-frequency oscillation (the oscillation frequency is 1.7 MHz or 2.4 MHz, which exceeds human hearing range, the electronic oscillations do no harm to humans or animals), through the high-frequency resonance of a ceramic atomizing sheet 3, the liquid water molecular structure is broken up to produce a naturally flowing water mist.

The heat source 5 heats the heating tube 4 so that the temperature of the heating tube 4 is controlled between 45-60 degrees. When the water mist enters the heating tube 4, the heating tube 4 can heat the water mist, and when heating, the temperature of the water mist is controlled between 45-60 degrees, which is within the acceptable temperature range of human skin, and will not be scalded.

In this embodiment, the heat source 5 may be a heating film, a heating wire, or MCH and other heating materials. Such the heat source 5 is energy-saving, environmentally friendly, and low-carbon, because the steaming face instrument on the market uses power of 200-300 W and has high power consumption. The steaming face device instrument of this embodiment has a power consumption of less than 20 W and saves energy by 90%.

The heating tube 4 is made of a metal part that can conduct heat, which can be a cylinder or other metal parts with regular shapes, such as a shape of a rectangular, a cube, or a conical. It should be noted that the heating tube 4 must be hollow and water mist will not be blocked when passing through the heating tube 4, and then can be smoothly ejected from the nozzle. In this embodiment, the length of the heating tube 4 is 20-30 mm, and the diameter is about 10 mm, so that the volume of the steaming face instrument can be controlled, which is convenient for portable use.

When the heat source 5 heats the heating tube 4, it is necessary to use heat insulation means for heat insulation to avoid burns. Therefore, the heating pipe 4 is arranged inside the heat insulation channel 2. One end of the heat insulation channel 2 is connected to the water outlet 12 and another end is connected to the nozzle of the steaming face instrument, and the water outlet 12, the atomizing sheet 3, the heat insulation channel 2 and the heating tube 4 are all coaxially arranged and arranged in order to ensure water mist is unobstructed, and the water mist comes out of the atomizing sheet 3 and enters the heating tube 4 very close to the atomizing sheet 3 for heating.

In summary, the disclosure includes the water delivery channel 1, the water delivery channel 1 includes the water inlet 11 and the water outlet 12; the atomizing sheet 3, the atomizing sheet is provided at the water outlet to water atomizing; and a heating mechanism connected to the water outlet 12 for heating the water mist to control the temperature of the water mist between 45 and 60 degrees. By adopting the above design, the disclosure enables the liquid water to be atomized by the high-frequency resonance of the atomizing sheet 3, and then heats the water mist, so that the temperature of the water mist is controlled at 45-60 degrees, which is safe and effective, and the volume of the steaming face instrument is greatly reduced, which is convenient to carry, and has low power consumption, energy saving and environmental protection.

The above-mentioned embodiments only express one or more embodiments of the disclosure, and their descriptions are more specific and detailed, but they should not be interpreted as limiting the scope of the disclosure. It should be noted that for those of ordinary skill in the art, without departing from the concept of the disclosure, a number of modifications and improvements can also be made, which all fall within the protection scope of the disclosure. Therefore, the protection scope of the disclosure shall be subject to the appended claims.

What is claimed is:

1. An atomizing device for a steaming face instrument, comprising:
a water delivery channel, which includes a water inlet and a water outlet;
an atomizing sheet provided at the water outlet for atomizing water; and
a heating mechanism, which is connected to the water outlet, is used to heat a water mist, so that a temperature of the water mist is controlled between 45 and 60 degrees.

2. The atomizing device of the steaming face instrument according to claim 1, wherein a groove is provided on the water outlet, the atomizing sheet is provided at the groove, the atomizing sheet and the water outlet are arranged coaxially.

3. The atomizing device of the steaming face instrument according to claim 1, wherein the atomizing sheet is an ultrasonic atomizing sheet.

4. The atomizing device of the steaming face instrument according to claim 2, wherein the heating mechanism comprises:
an insulating channel, a heating tube and a heat source, and one end of the insulating channel is connected to the water outlet, and the atomizing sheet is fixed at the water outlet, another end is connected to a spout, the heating tube is arranged in the insulation channel and is coaxial with the water outlet, and the heat source surrounds an outer wall of the heating tube.

5. The atomizing device of the steaming face instrument according to claim 4, wherein a convex ring is provided at an end of the insulation channel connected to the water outlet, and the convex ring cooperates with the groove to fix the atomizing sheet, and the convex ring and the water outlet are arranged coaxially.

6. The atomizing device of the steaming face instrument according to claim 5, wherein the heating tube is made of metal, and a length of the heating tube is 20-30 mm.

7. The atomizing device of the steaming face instrument according to claim 6, wherein the heat source is a heating film or a heating wire.
